(19) United States
(12) Patent Application Publication
(10) Pub. No.: US 2026/0191484 A1
(43) Pub. Date: Jul. 9, 2026

(54) SYSTEMS AND METHODS FOR DOSE MODULATION OF X-RAY SCANNING

(71) Applicant: SHANGHAI UNITED IMAGING HEALTHCARE CO., LTD., Shanghai (CN)

(72) Inventors: Fan ZHAO, Shanghai (CN); Guoping ZHU, Shanghai (CN); Bin CAO, Shanghai (CN)

(73) Assignee: SHANGHAI UNITED IMAGING HEALTHCARE CO., LTD., Shanghai (CN)

(21) Appl. No.: 19/557,034

(22) Filed: Mar. 4, 2026

Related U.S. Application Data
(63) Continuation of application No. PCT/CN2024/119995, filed on Sep. 20, 2024.

(30) Foreign Application Priority Data
Sep. 20, 2023 (CN) 202311222500.5

(51) Int. Cl. A61B 6/40; A61B 6/00; A61B 6/03

(52) U.S. Cl. CPC A61B 6/405; A61B 6/032; A61B 6/54

(57) ABSTRACT

The present disclosure provides a system and method for dose modulation of an X-ray scanning. The method may include obtaining an initial scanning dose rule for scanning a target subject; determining, based on the initial scanning dose rule, a power supply control rule of a grid in an X-ray tube of the X-ray scanning; and controlling a high-voltage generator to supply power to the grid according to the power supply control rule to control an output state of tube current of the X-ray tube.

### CROSS-REFERENCE TO RELATED APPLICATIONS

This application is a continuation of International Patent Application No. PCT/CN2024/119995, filed on September 20, 2024, which claims the priority of Chinese Patent Application No. 202311222500.5, filed on September 20, 2023, the contents of each of which are hereby incorporated by reference.

### TECHNICAL FIELD

The disclosure generally relates to the field of X-ray scanning, and more particularly relates to systems and methods for dose modulation of X-ray scanning.

### BACKGROUND

For X-ray scanning systems, while meeting the needs of imaging/therapy, the radiation dose should be minimized as much as possible to protect the radiation safety of patients and medical workers, that is, to follow the principle of as low as reasonably achievable (ALARA). During a scanning process, when a relative position (or a scanning position) of a radiation source and a scanned subject (e.g., a patient) changes, i.e., an equivalent scanned load characteristic (e.g., an equivalent thickness of the scanned subject) changes, the X-ray energy detected by a detector may change accordingly. In order to follow the ALARA principle and meet the conditions of image brightness and signal-to-noise ratio at different positions, it is necessary to dynamically adjust the radiation dose. Specifically, when the equivalent thickness decreases, the dose should be decreased, and when the equivalent thickness increases, the dose should be increased, thereby reducing the total radiation dose throughout the entire scanning process.

Therefore, it is desired to provide systems and methods for dose modulation of an X-ray scanning, so that the radiation dose can quickly and accurately follow changes in the target dose.

### SUMMARY

According to a first aspect of the present disclosure, a system is provided. The system may include at least one storage device storing executable instructions, and at least one processor in communication with the at least one storage device. When executing the executable instructions, the at least one processor may cause the system to perform operations including: obtaining, for scanning a target subject, an acquisition cycle of a detector and an initial scanning dose curve; generating a tube current modulation carrier wave at least based on the acquisition cycle and a target tube current, the target tube current being greater than or equal to a maximum tube current in the initial scanning dose curve; determining, based on the tube current modulation carrier wave and the initial scanning dose curve, a power supply control rule of a grid in an X-ray tube of the X-ray scanning; and controlling a high-voltage generator to supply power to the grid according to the power supply control rule to control an output state of tube current of the X-ray tube, wherein an output value of the tube current is equal to the target tube current.

In some embodiments, the tube current modulation carrier wave is a sawtooth wave including a plurality of serrations, each serration being in a triangular shape in each acquisition cycle.

In some embodiments, the triangular shape is a right triangle or an isosceles triangle.

In some embodiments, when the triangular shape is the right triangle and a reset time of the detector is equal to 0, an absolute value of a slope of an inclination edge of the each serration is equal to a ratio of the target tube current to the corresponding acquisition cycle.

In some embodiments, when the triangular shape is the isosceles triangle and a reset time of the detector is equal to 0, a slope of a front edge and an absolute value of a slope of a rear edge of the each serration are both equal to a ratio of the target tube current to half of the corresponding acquisition cycle.

In some embodiments, the controlling a high-voltage generator to supply power to the grid of the X-ray tube according to the power supply control rule includes: during a first target period of each acquisition cycle, controlling the high-voltage generator to provide a first level to the grid to turn off the grid; and during a second target period of the acquisition cycle, controlling the high-voltage generator to provide a second level to the grid to turn on the grid, wherein the first target period corresponds to a period in the acquisition cycle in which an amplitude of the initial scanning dose curve is greater than or equal to an amplitude of the tube current modulation carrier wave, and the second target period corresponds to a period in the acquisition cycle in which an amplitude of the initial scanning dose curve is less than an amplitude of the tube current modulation carrier wave.

In some embodiments, when the triangular shape is the right triangle and a reset time of the detector is not equal to 0, an absolute value of a slope of an edge of the each serration is equal to a ratio of the target tube current to a difference between the corresponding acquisition cycle and the reset time.

In some embodiments, when the triangular shape is the isosceles triangle and a reset time of the detector is not equal to 0, a slope of a front edge and an absolute value of a slope of a rear edge of the each serration are both equal to a ratio of the target tube current to half of a difference between the acquisition cycle and the reset time.

In some embodiments, the controlling a high-voltage generator to supply power to the grid of the X-ray tube according to the power supply control rule includes: during a third target period of each acquisition cycle, controlling the high-voltage generator to provide a first level to the grid to turn off the grid; and during a fourth target period of the acquisition cycle, controlling the high-voltage generator to provide a second level to the grid to turn on the grid, wherein the third target period is a difference between a first time period and the reset time of the detector, wherein the first target period corresponds to a period in the acquisition cycle in which an amplitude of the initial scanning dose curve is greater than or equal to an amplitude of the tube current modulation carrier wave, and the fourth target period is a sum of a second target period and the reset time of the detector, wherein the second target period corresponds to a period in the acquisition cycle in which an amplitude of the initial scanning dose curve is less than an amplitude of the tube current modulation carrier wave.

In some embodiments, the controlling a high-voltage generator to supply power to the grid of the X-ray tube according to the power supply control rule includes: recording a power operation status of the high-voltage generator of the X-ray tube over time; and after a target dose of X-ray scanning corresponding to a current emission time period is provided to the target subject, determining a target switching time point for controlling the high-voltage generator to turn on the grid based on the power operation status, the current emission time period is determined based on the power supply control rule.

In some embodiments, when the high-voltage generator operates in a discontinuous current mode, the target switching time point corresponds to a time point when a resonant inductor current in the high-voltage generator is 0.

In some embodiments, the at least one processor is further configured to cause the system to perform operations including: simultaneously recording a power modulation status of the high-voltage generator at the target switching time point; and controlling the high-voltage generator to turn off the grid at a first switching time point by providing the power modulation status of the high-voltage generator at the target switching time point to the high-voltage generator, wherein the first switching time point is a time point corresponding to a starting point of an emission time period next to the current emission time period for controlling the high-voltage generator to turn off the grid.

In some embodiments, when the high-voltage generator operates in a continuous current mode, the target switching time point corresponds to a time point after the target dose of the specific time period is provided to the target subject.

In some embodiments, the at least one processor is further configured to cause the system to perform operations including: simultaneously recording a power modulation status and a target resonant inductor current of the high-voltage generator at the target switching time point; and controlling the high-voltage generator to turn off the grid at a second switching time point by providing the power modulation status of the high-voltage generator at the switching time point to the high-voltage generator, wherein the second switching time point is a time point when a resonant inductor current of the high-voltage generator is equal to the target resonant inductor current that is after a starting point of an emission time target period next to the current emission time period for controlling the high-voltage generator to turn off the grid.

According to a second aspect of the present disclosure, a method for dose modulation of an X-ray scanning is provided. The method may be implemented on at least one computing device, each of which may include at least one processor and a storage device. The method may include: obtaining, for scanning a target subject, an acquisition cycle of a detector and an initial scanning dose curve; generating a tube current modulation carrier wave at least based on the acquisition cycle and a target tube current, the target tube current being greater than or equal to a maximum tube current in the initial scanning dose curve; determining, based on the tube current modulation carrier wave and the initial scanning dose curve, a power supply control rule of a grid in an X-ray tube of the X-ray scanning; and controlling a high-voltage generator to supply power to the grid according to the power supply control rule to control an output state of tube current of the X-ray tube, wherein an output value of the tube current is equal to the target tube current.

According to a third aspect of the present disclosure, a non-transitory computer-readable medium storing at least one set of instructions is provided. When executed by at least one processor, the at least one set of instructions may direct the at least one processor to perform a method. The method may include: obtaining, for scanning a target subject, an acquisition cycle of a detector and an initial scanning dose curve; generating a tube current modulation carrier wave at least based on the acquisition cycle and a target tube current, the target tube current being greater than or equal to a maximum tube current in the initial scanning dose curve; determining, based on the tube current modulation carrier wave and the initial scanning dose curve, a power supply control rule of a grid in an X-ray tube of the X-ray scanning; and controlling a high-voltage generator to supply power to the grid according to the power supply control rule to control an output state of tube current of the X-ray tube, wherein an output value of the tube current is equal to the target tube current.

According to a fourth aspect of the present disclosure, a system is provided. The system may include at least one storage device storing executable instructions, and at least one processor in communication with the at least one storage device. When executing the executable instructions, the at least one processor may cause the system to perform operations including: obtaining a power supply control rule for controlling a high-voltage generator; determining a first initial switching time point for switching a high-voltage generator from a working load state to a no-load state based on the power supply control rule, if the high-voltage generator operates in a discontinuous current mode, controlling the high-voltage generator to switch from the working load state to the no-load state at a first target switching time point when a resonant inductor current in the high-voltage generator is 0; or if the high-voltage generator operates in a continuous current mode, controlling the high-voltage generator to switch from the working load state to the no-load state at the first initial switching time point.

In some embodiments, the at least one processor is further configured to cause the system to perform operations including: determining a second initial switching time point for switching the high-voltage generator from the no-load state to the working load state based on the power supply control rule, if the high-voltage generator operates in the discontinuous current mode, obtaining a power modulation status of the high-voltage generator at the first target switching time point, and controlling the high-voltage generator to switch from the no-load state to the working load state at the second initial switching time point by providing the power modulation status of the high-voltage generator at the first target switching time point to the high-voltage generator; or if the high-voltage generator operates in the continuous current mode, obtaining a power modulation status and a target resonant inductor current of the high-voltage generator at the first initial switching time point, and controlling the high-voltage generator to switch from the no-load state to the working load state at a second target switching time point by providing the power modulation status of the high-voltage generator at the first initial switching time point to the high-voltage generator, wherein the second target switching time point corresponds to a time point when the resonant inductor current of the high-voltage generator is equal to the target resonant inductor current of the high-voltage generator at the first initial switching time point.

According to a fifth aspect of the present disclosure, a method for power adjustment is provided. The method may be implemented on at least one computing device, each of which may include at least one processor and a storage device. The method may include: obtaining a power supply control rule for controlling a high-voltage generator; determining a first initial switching time point for switching a high-voltage generator from a working load state to a no-load state based on the power supply control rule, if the high-voltage generator operates in a discontinuous current mode, controlling the high-voltage generator to switch from the working load state to the no-load state at a first target switching time point when a resonant inductor current in the high-voltage generator is 0; or if the high-voltage generator operates in a continuous current mode, controlling the high-voltage generator to switch from the working load state to the no-load state at the first initial switching time point.

According to a sixth aspect of the present disclosure, a non-transitory computer-readable medium storing at least one set of instructions is provided. When executed by at least one processor, the at least one set of instructions may direct the at least one processor to perform a method. The method may include: obtaining a power supply control rule for controlling a high-voltage generator; determining a first initial switching time point for switching a high-voltage generator from a working load state to a no-load state based on the power supply control rule, if the high-voltage generator operates in a discontinuous current mode, controlling the high-voltage generator to switch from the working load state to the no-load state at a first target switching time point when a resonant inductor current in the high-voltage generator is 0; or if the high-voltage generator operates in a continuous current mode, controlling the high-voltage generator to switch from the working load state to the no-load state at the first initial switching time point.

According to a seventh aspect of the present disclosure, a system is provided. The system may include at least one storage device storing executable instructions, and at least one processor in communication with the at least one storage device. When executing the executable instructions, the at least one processor may cause the system to perform operations including: obtaining an initial scanning dose rule for scanning a target subject; determining, based on the initial scanning dose rule, a power supply control rule of a grid in an X-ray tube of the X-ray scanning; and controlling a high-voltage generator to supply power to the grid according to the power supply control rule to control an output state of tube current of the X-ray tube.

In some embodiments, the power supply control rule is configured to maintain an output value of the tube current, and change an emission dose of each acquisition cycle of a detector by controlling an emission time period through turning on/off the grid.

In some embodiments, the initial scanning dose rule includes an initial scanning dose curve or a plurality of discrete points.

In some embodiments, the initial scanning dose rule is the initial scanning dose curve, the determining, based on the initial scanning dose rule, a power supply control rule of a grid in an X-ray tube of the X-ray scanning includes: generating a tube current modulation carrier wave at least based on an acquisition cycle of the detector and a target tube current, the target tube current being greater than or equal to a maximum tube current in the initial scanning dose curve; and determining, based on the tube current modulation carrier wave and the initial scanning dose curve, the power supply control rule.

In some embodiments, the controlling a high-voltage generator to supply power to the grid according to the power supply control rule to control an output state of tube current of the X-ray tube includes: controlling the high-voltage generator to supply power to the grid according to the power supply control rule to control the output state of tube current of the X-ray tube, wherein an output value of the tube current is equal to the target tube current.

According to an eighth aspect of the present disclosure, a method for dose modulation of an X-ray scanning is provided. The method may be implemented on at least one computing device, each of which may include at least one processor and a storage device. The method may include: obtaining an initial scanning dose rule for scanning a target subject; determining, based on the initial scanning dose rule, a power supply control rule of a grid in an X-ray tube of the X-ray scanning; and controlling a high-voltage generator to supply power to the grid according to the power supply control rule to control an output state of tube current of the X-ray tube.

According to a ninth aspect of the present disclosure, a non-transitory computer-readable medium storing at least one set of instructions is provided. When executed by at least one processor, the at least one set of instructions may direct the at least one processor to perform a method. The method may include: obtaining an initial scanning dose rule for scanning a target subject; determining, based on the initial scanning dose rule, a power supply control rule of a grid in an X-ray tube of the X-ray scanning; and controlling a high-voltage generator to supply power to the grid according to the power supply control rule to control an output state of tube current of the X-ray tube.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not scaled. These embodiments are non-limiting exemplary embodiments, in which like reference numerals represent similar structures throughout the several views of the drawings, and wherein:

FIG. 1 is a schematic diagram illustrating an exemplary imaging system 100 according to some embodiments of the present disclosure;

FIG. 2 is a schematic diagram illustrating exemplary curves related to dose modulation of an X-ray scanning according to some embodiments of the present disclosure;

FIG. 3 is a block diagram illustrating an exemplary processing device according to some embodiments of the present disclosure;

FIG. 4A is a flowchart illustrating an exemplary process for dose modulation of an X-ray scanning according to some embodiments of the present disclosure;

FIG. 4B is a diagram illustrating an exemplary tube current modulation carrier wave according to some embodiments of the present disclosure;

FIG. 4C is a flowchart illustrating an exemplary process for dose modulation of an X-ray scanning according to some embodiments of the present disclosure;

FIG. 5A is a schematic diagram illustrating an exemplary process for generating a grid power supply control rule when the target tube current is equal to the maximum tube current in the initial scanning dose curve according to some embodiments of the present disclosure;

FIG. 5B is a schematic diagram illustrating an exemplary process for generating a grid power supply control rule when the target tube current is equal to the maximum tube current in the initial scanning dose curve according to some embodiments of the present disclosure;

FIG. 5C is a schematic diagram illustrating an exemplary process for generating a grid power supply control rule when the target tube current is equal to the maximum tube current in the initial scanning dose curve according to some embodiments of the present disclosure;

FIG. 6 is a diagram illustrating exemplary variation curves of tube voltage and tube current when turning on/off a grid according to some embodiments of the present disclosure;

FIG. 7 is a flowchart illustrating an exemplary process for controlling a high-voltage generator to supply power to a grid according to a power supply control rule according to some embodiments of the present disclosure;

FIG. 8A is a diagram illustrating a resonant inductor current curve and a resonant capacitor voltage curve of a high-voltage generator when the high-voltage generator operates in a discontinuous current mode according to some embodiments of the present disclosure; and

FIG. 8B is a diagram illustrating a resonant inductor current curve and a resonant capacitor voltage curve of a high-voltage generator when the high-voltage generator operates in a continuous current mode according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including” when used in this disclosure, specify the presence of stated features, integers, steps, operations, elements, and/or components, but do not preclude the presence or addition of one or more other features, integers, steps, operations, elements, components, and/or groups thereof.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments in the present disclosure. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

Generally, the manner for dose modulation is to maintain a tube voltage of the X-ray tube constant and change the X-ray dose emitted by the X-ray tube by adjusting the tube current during the scanning process. Since the magnitude of the tube current depends on a filament temperature of the X-ray tube, the purpose of adjusting the tube current is achieved by adjusting the filament temperature. However, there is a problem of slow adjustment speed of filament temperature, usually reaching tens or even hundreds of milliseconds. Thus, the aforementioned manner for dose modulation may result in the inability of the tube current to quickly track a target dose and change accordingly, which not only fails to effectively reduce the radiation dose, but may also cause insufficient image brightness and signal-to-noise ratio when the equivalent thickness of the scanned subject is thick.

Provided herein are systems and methods for dose modulation of an X-ray scanning. For example, the method may include obtaining, for scanning a target subject, an acquisition cycle of a detector and an initial scanning dose curve. The method may further include generating a tube current modulation carrier wave at least based on the acquisition cycle, the initial scanning dose curve, and a target tube current. The target tube current may be greater than or equal to a maximum tube current in the initial scanning dose curve. The method may further include determining, based on the tube current modulation carrier wave and the initial scanning dose curve, a power supply control rule of a grid in an X-ray tube of the X-ray scanning, and controlling a high-voltage generator to supply power to the grid according to the power supply control rule to control an output state of tube current of the X-ray tube. An output value of the tube current may be equal to the target tube current.

Accordingly, the dose modulation of the X-ray scanning can be controlled through the grid using the power supply control rule of the grid, that is, to control the emission time of the X-ray tube at different scanning positions while keeping an output amplitude of the tube current unchanged to achieve dose modulation, thereby avoiding real-time changes in a filament temperature of the X-ray tube during the scanning process. Moreover, since a control speed of the grid is generally in microseconds or tens of microseconds, which has an order of magnitude improvement in control effect compared to filament temperature control, so that the radiation dose can quickly and accurately follow changes in a target dose. Moreover, the output current of the X-ray tube is equal to the target tube current which is greater than or equal to a maximum tube current in the initial scanning dose curve, which can meet the requirements of image brightness and signal-to-noise ratio of the target subject at different scanning positions (or scanning angles).

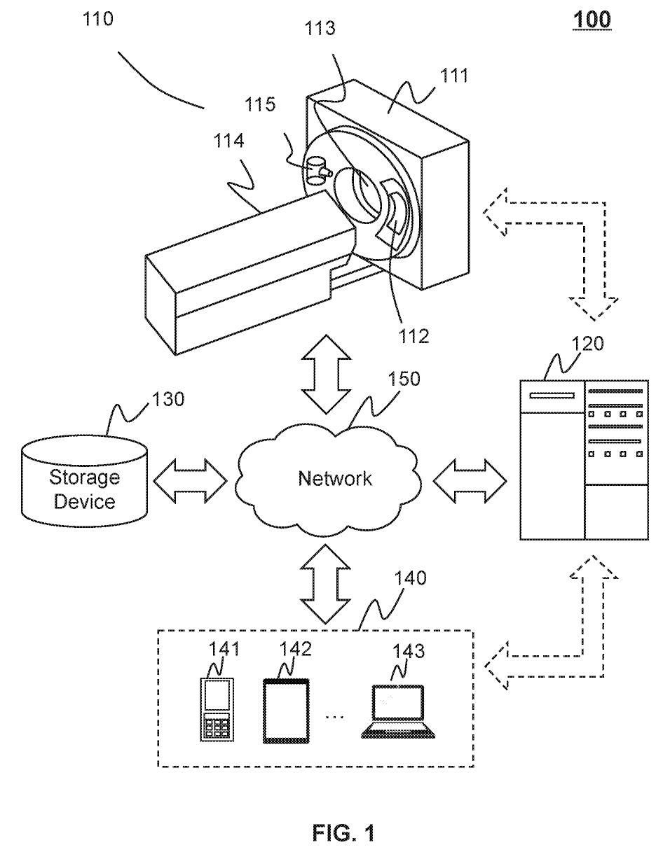

FIG. 1 is a schematic diagram illustrating an exemplary imaging system 100 according to some embodiments of the present disclosure. In some embodiments, the imaging system 100 may be applied to any application scenario in which radiation rays (e.g., X-rays) are used for generating images and/or providing treatment, such as a computed tomography (CT) system, a digital radiography (DR) system, a C-arm X-ray system, a computed tomography-positron emission tomography (CT-PET) system, an image-guide radiotherapy (IGRT) system (e.g., a CT guided radiotherapy system), or the like, or a combination thereof.

As illustrated in FIG. 1, the imaging system 100 may include an imaging device 110, a processing device 120, a storage device 130, a terminal device 140, and a network 150. The components in the imaging system 100 may be connected to and/or communicate with each other via a wireless connection, a wired connection, or a combination thereof.

The imaging device 110 may be configured to scan a subject using radiation rays and generate imaging data used to generate one or more images relating to the subject. In some embodiments, the imaging device 110 may include a computed tomography (CT) scanner, a digital radiography (DR) scanner, a C-arm X-ray scanner, a digital subtraction angiography (DSA) scanner, a dynamic spatial reconstructor (DSR) scanner, an X-ray microscopy scanner, a multi-modality scanner, or the like, or a combination thereof. Exemplary multi-modality scanners may include a computed tomography-positron emission tomography (CT-PET) scanner, a computed tomography-magnetic resonance imaging (CT-MRI) scanner, etc.

The imaging device 110 may include a gantry 111, one or more detectors 112, a detecting region 113, a table 114, a radiation source 115, or any other component. The gantry 111 may be configured to provide support for other components (e.g., the radiation source 115, the detector(s) 112, etc.) of the imaging device 110. In some embodiments, the detector(s) 112 and the radiation source 115 may be oppositely mounted on the gantry 111. In some embodiments, the gantry 111 may rotate and/or move. The detector(s) 112 and the radiation source 115 may rotate along with the rotation of the gantry 111. The table 114 may be configured to locate and/or support a scanned subject. A scanned subject may be placed on the table 114 and moved into the detecting region 113 (e.g., a space between the detector(s) 112 and the radiation source 115) of the imaging device 110. The scanned subject may be biological or non-biological. Merely by way of example, the scanned subject may include a patient, a man-made object, etc. As another example, the scanned subject may include a specific portion, organ, and/or tissue of the patient. For example, the scanned subject may include head, brain, neck, body, shoulder, arm, thorax, heart, stomach, blood vessel, soft tissue, knee, feet, or the like, or any combination thereof.

The radiation source 115 may be configured to generate and/or emit radiation rays (e.g., X-rays) to scan the scanned subject that is placed on the table 114. In some embodiments, the radiation source 115 may include a high-voltage generator, a tube (e.g., an X-ray tube), or any other components (e.g., a collimator). The high-voltage generator may be configured to provide a voltage and/or current for the tube, and/or provide power for other components (e.g., a cathode filament) of the radiation source 115. The tube may be a vacuum diode that operates at high voltage. The tube may be configured to generate radiation rays when a high-voltage is applied to the tube by the high-voltage generator. As used herein, the voltage applied to the tube may be also referred to as a tube voltage. In some embodiments, the tube may include a cathode filament and an anode target. The voltage generated by the high-voltage generator may trigger the cathode filament to emit a plurality of electrons to form an electron beam (also referred as a tube current). The emitted electron beam may be impinged on a small area (i.e., a focus) on the anode target to generate radiation beams (e.g., X-rays beams) consisting of high-energetic photons. In some embodiments, the radiation rays may include X-rays, γ-rays, α-rays, or the like, or a combination thereof.

In some embodiments, the radiation source 115 may further include a grid disposed at the cathode filament. The grid may be configured to control an output state of the tube current. Specifically, when the grid is turned on, the tube has no tube current output and does not emit radiation rays. When the grid is turned off, the tube has a tube current output and emits radiation rays.

The detector(s) 112 may detect the radiation beams penetrated through at least part of the scanned subject within the detection region 113. Specifically, the detector(s) 112 may detect the penetrated radiation beams periodically. In some embodiments, an acquisition cycle of the detector may be set according to a default setting of the imaging system 100 or preset by a user (e.g., a doctor or an operator) via the terminal device 140. In some embodiments, the detector(s) 112 may include a plurality of detector units, which may be arranged in any suitable manner, for example, a channel direction and a row direction. The detector(s) 112 may include a scintillation detector (e.g., a cesium iodide detector), a gas detector, etc.

The processing device 120 may process data and/or information relating to dose modulation to perform one or more functions described in the present disclosure. For example, the processing device 120 may generate a tube current modulation carrier wave at least based on an acquisition cycle of the detector 112, an initial scanning dose curve for scanning a target subject, and a target tube current of the tube, wherein the target tube current is greater than or equal to a maximum tube current in the initial scanning dose curve. The processing device 120 may control the X-ray tube (or the high-voltage generator) to modulate the dose for scanning the target subject based on the tube current modulation carrier wave and the initial scanning dose curve. Further, the processing device 120 may adjust the high-voltage generator of the tube based on a power operation status of the high-voltage generator. In some embodiments, the processing device 120 may be a computer, a user console, a single server or a server group, etc. The server group may be centralized or distributed. In some embodiments, the processing device 120 may be local or remote. In some embodiments, the processing device 120 may be implemented on a cloud platform. In some embodiments, the processing device 120 may be implemented on a computing device or the imaging device 110.

The storage device 130 may be configured to store data and/or instructions. The data and/or instructions may be obtained from, for example, the processing device 120, the imaging device 110, and/or any other component of the imaging system 100. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or any combination thereof. In some embodiments, the storage device 130 may be implemented on a cloud platform.

The terminal device 140 may be configured to receive information and/or data from the processing device 120, the imaging device 110, and/or the storage device 130 via the network 150. For example, the terminal device 140 may receive an image from the processing device 120. In some embodiments, the terminal device 140 may provide a user interface via which a user may view information and/or input data and/or instructions to the imaging system 100. For example, the user may view, via the user interface, information associated with the imaging device 110. As another example, the user may input, via the user interface, a user input instruction to set an acquisition cycle of the detector 112. In some embodiments, the terminal device 140 may include a mobile device 141, a tablet computer 142, a laptop computer 143, or the like, or any combination thereof. In some embodiments, the terminal device 140 may include a display that can display information in a human-readable form, such as text, image, audio, video, graph, animation, or the like, or any combination thereof.

The network 150 may facilitate the exchange of information and/or data for the imaging system 100. In some embodiments, one or more components (e.g., the imaging device 110, the processing device 120, the terminal device 140, or the storage device 130) of the imaging system 100 may transmit information and/or data to one or more other components of the imaging system 100 via the network 150. In some embodiments, the network 150 may be any type of wired or wireless network, or combination thereof.

It should be noted that the above description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. In some embodiments, the imaging system 100 may include one or more additional components and/or one or more components described above may be omitted. Merely by way of example, the imaging system 100 may not include the terminal device 140. Additionally or alternatively, two or more components of the imaging system 100 may be integrated into a single component. For example, the processing device 120 may be integrated into the imaging device 110. As another example, a component of the imaging system 100 may be replaced by another component that can implement the functions of the component. However, those variations and modifications do not depart from the scope of the present disclosure.

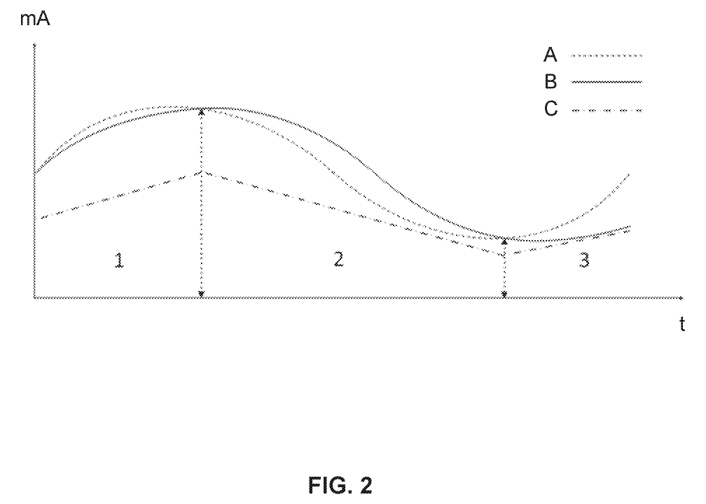

FIG. 2 is a schematic diagram illustrating exemplary curves related to dose modulation of an X-ray scanning according to some embodiments of the present disclosure. As shown in FIG. 2, a curve A represents a curve of tube current (mA) required for dose modulation over time (t). A curve B represents a curve of the actual output tube current (mA) over time (t). A curve C represents a dynamic variation curve of filament current during dose modulation. From FIG. 2, it can be seen that in zones 1 and 3, the actual output tube current is less than the tube current required for dose modulation, which may lead to a decrease in the brightness and signal-to-noise ratio of medical images. In zone 2, the actual output tube current is higher than the tube current required for dose modulation, which may lead to a higher output dose and failure to follow the ALARA principle.

To solve the above problems, for an X-ray scanning process of an imaging device, the embodiments of the present disclosure provide a system and a method for dose modulation of the X-ray scanning, which maintains the amplitude of the tube current of the X-ray tube unchanged and changes an output time of the tube current (i.e. an emission time of the X-ray tube) during each image acquisition cycle of the imaging device to achieve the purpose of dose modulation.

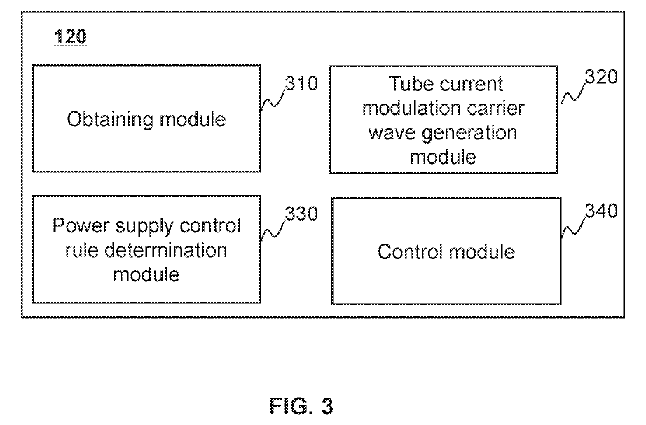

FIG. 3 is a block diagram illustrating an exemplary processing device according to some embodiments of the present disclosure. As illustrated in FIG. 3, the processing device 120 may include an obtaining module 310, a tube current modulation carrier wave generation module 320, a power supply control rule determination module 330, and a control module 340.

The obtaining module 310 may be configured to obtain data and/or information for dose modulation. For example, the obtaining module 310 may obtain, for scanning a target subject, an acquisition cycle of a detector and an initial scanning dose curve. In some embodiments, for power adjustment of a high-voltage generator, the obtaining module 310 may obtain a power supply control rule for controlling a high-voltage generator.

The tube current modulation carrier wave generation module 320 may be configured to generate a tube current modulation carrier wave at least based on the acquisition cycle and a target tube current. The target tube current may be greater than or equal to a maximum tube current in the initial scanning dose curve.

The power supply control rule determination module 330 may be configured to determine, based on the tube current modulation carrier wave and the initial scanning dose curve, a power supply control rule of a grid in an X-ray tube of the X-ray scanning. In some embodiments, the power supply control rule determination module 330 may determine a first initial switching time point for switching a high-voltage generator from a working load state to a no-load state based on the power supply control rule.

The control module 340 may be configured to control a high-voltage generator to supply power to the grid according to the power supply control rule to control an output state of tube current of the X-ray tube. An output value of the tube current is equal to the target tube current. In some embodiments, for power adjustment of a high-voltage generator, if the high-voltage generator operates in a discontinuous current mode, the control module 340 may control the high-voltage generator to switch from the working load state to the no-load state at a first target switching time point when a resonant inductor current in the high-voltage generator is 0. If the high-voltage generator operates in a continuous current mode, the control module 340 may control the high-voltage generator to switch from the working load state to the no-load state at the first initial switching time point.

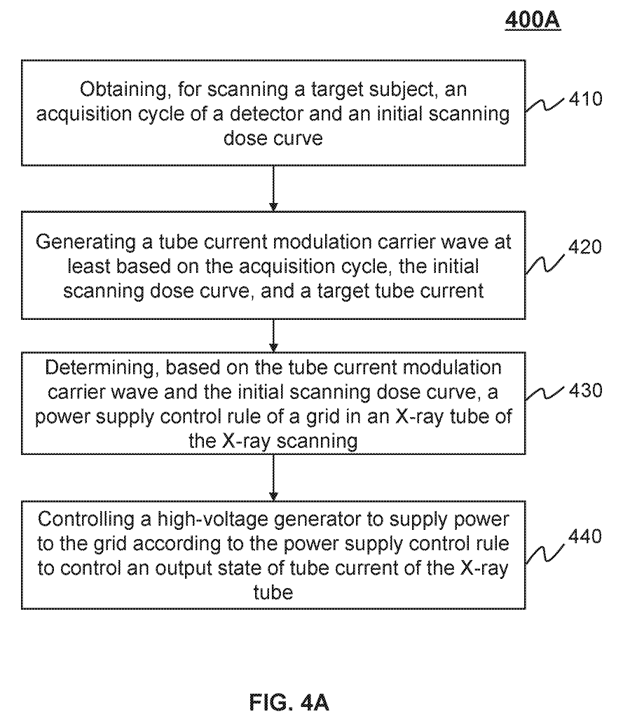

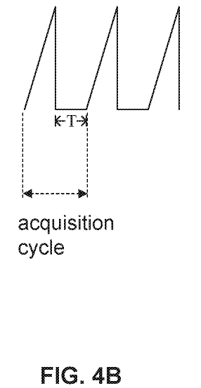

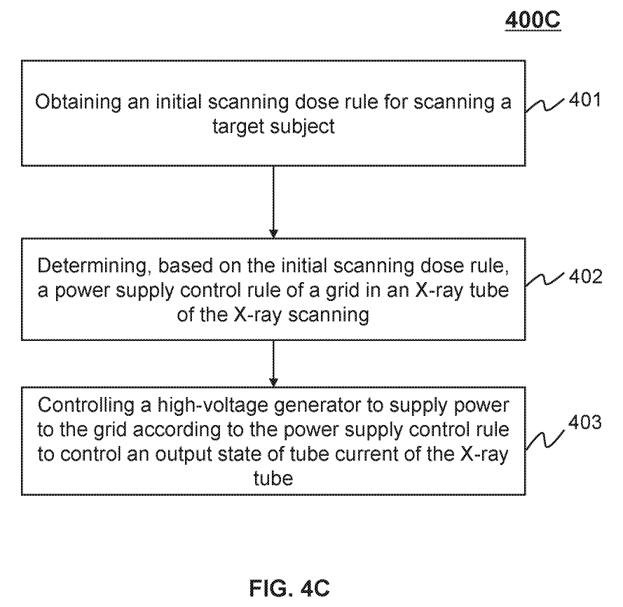

More descriptions about the dose modulation and the power adjustment may be found elsewhere in the present disclosure (e.g., FIG. 4A and FIG. 7 and the descriptions thereof).

It should be noted that the above description is merely provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 120 may include one or more additional modules, such as a storage module (not shown) for storing data. In some embodiments, the modules in the processing device 120 may be connected to or communicate with each other via a wired connection or a wireless connection. In some embodiments, two or more of the modules may be combined as a single module, and any one of the modules may be divided into two or more units.

FIG. 4A is a flowchart illustrating an exemplary process for dose modulation of an X-ray scanning according to some embodiments of the present disclosure. In some embodiments, process 400A may be executed by the imaging system 100. For example, process 400A may be implemented as a set of instructions (e.g., an application) stored in the storage device 130, and the processing device 120 (e.g., one or more modules in FIG. 3) may execute the set of instructions and may accordingly be directed to perform the process 400A.

In 410, the processing device 120 (e.g., the obtaining module 310) may obtain, for scanning a target subject, an acquisition cycle of a detector and an initial scanning dose curve.

An imaging device (e.g., the imaging device 110) may be used to scan the target subject. As described in connection with FIG. 1, the imaging device may include the detector and an X-ray tube.

The acquisition cycle of the detector refers to a time required to acquire one image frame. In some embodiments, the acquisition cycle of the detector may be determined by dividing a time required for a gantry of the imaging device to rotate one circle by a count of image frames acquired during the time. In some embodiments, the acquisition cycle of the detector may be set according to a default setting of the detector or preset by a user (e.g., a doctor or an operator) via the terminal device 140.

Generally, when a relative position between the X-ray tube and the target subject changes (e.g., a scanning angle changes), an equivalent thickness of the target subject (i.e., a thickness of the portion of the target subject that X-rays pass through) may change, resulting in a corresponding change in an X-ray energy (or a radiation dose) detected by the detector and/or a radiation dose received by the target subject. Thus, during a process for scanning the target subject, since the movement of the target subject with respect to the X-ray tube (or the detector), in order to follow the ALARA principle, for different relative positions between the X-ray tube and the target subject, the radiation dose emitted by the X-ray tube may vary, that is, the tube current of the X-ray tube may vary over time during the scanning process.

In the present disclosure, a curve representing the variation of the tube current of the X-ray tube required for scanning the target subject over time can be the initial scanning dose curve. The initial scanning dose curve may characterize a corresponding relationship between the tube current of the X-ray tube and an emission time of the X-ray tube during the scanning process. It should be noted that although the variation of the tube current over time described in the present disclosure is represented as a curve, other representations of the variation of the tube current over time do not depart from the scope of the present disclosure. For example, the variation of the tube current over time may also be represented as a table, a function, discrete points, etc. The initial scanning dose curve may be determined based on the table, the function, the discrete points, etc. For example, the initial scanning dose curve may be obtained by fitting the discrete points.

In some embodiments, the tube current in the initial scanning dose curve may be positively correlated with an equivalent thickness of the target subject. That is to say, when scanning a relatively thick position of the target subject, the radiation dose should be increased, while scanning a relatively thin position of the target subject, the radiation dose should be decreased. Specifically, for scanning the target subject, a maximum tube current in the initial scanning dose curve may be positively correlated with a maximum equivalent thickness of the target subject. Merely by way of example, if the target subject is a lung of a patient, during the scanning of the lung, the equivalent thickness of the lung may vary at different scanning angles around the patient, so the maximum tube current of the initial scanning dose curve is positively correlated with the maximum equivalent thickness of the lung at different scanning angles.

In some embodiments, the initial scanning dose curve may be determined based on a scan protocol associated with the target subject. Different target subjects may correspond to different scan protocols. For example, for the target subject including the head of a patient to be scanned, the scan protocol may be a protocol corresponding to a head scan. As another example, for the target subject including the chest of the patient to be scanned, the scan protocol may be a protocol corresponding to a chest scan. The processing device 120 may obtain the scan protocol from a storage device (e.g., the storage device 130) directly.

In some embodiments, the initial scanning dose curve may be determined based on a positioning image of the target subject. For example, for treating the target subject, before treating the target subject (i.e., performing the current scanning process with a significantly high dose of radiation) using X-rays, the imaging device may generate a positioning image of the target subject based on imaging data obtained by scanning the target subject using X-rays with a significantly low dose. Further, the processing device 120 may determine the initial scanning dose curve based on the generated positioning image of the target subject.

In some embodiments, the processing device 120 may obtain a corresponding relationship between the thickness of the target subject and the initial scanning dose curve by fitting experimental data and/or historical data containing two parameters of the thickness of the target subject and the initial scanning dose curve. During the scanning process, the processing device 120 may determine the initial scanning dose curve that matches the thickness of the target subject based on the corresponding relationship.

In 420, the processing device 120 (e.g., the tube current modulation carrier wave generation module 320) may generate a tube current modulation carrier wave at least based on the acquisition cycle and a target tube current.

The target tube current may be greater than or equal to a maximum tube current in the initial scanning dose curve.

FIG. 4B is a diagram illustrating an exemplary tube current modulation carrier wave according to some embodiments of the present disclosure. In some embodiments, the tube current modulation carrier wave may be determined based on an acquisition cycle, a reset time T of the detector and a target tube current. The reset time T of the detector is related to hardware performance of the detector.

The tube current modulation carrier wave may be a sawtooth wave including a plurality of serrations. Each serration may form a triangular shape with a horizontal line in each acquisition cycle. In the present disclosure, the shape formed by each serration with the horizontal line is also referred to as the shape of each serration. For each serration, a peak value of the serration (or the triangular shape) may be equal to the target tube current.

It should be noted that in some embodiments, the tube current modulation carrier wave may also be represented as a table, a function, etc.

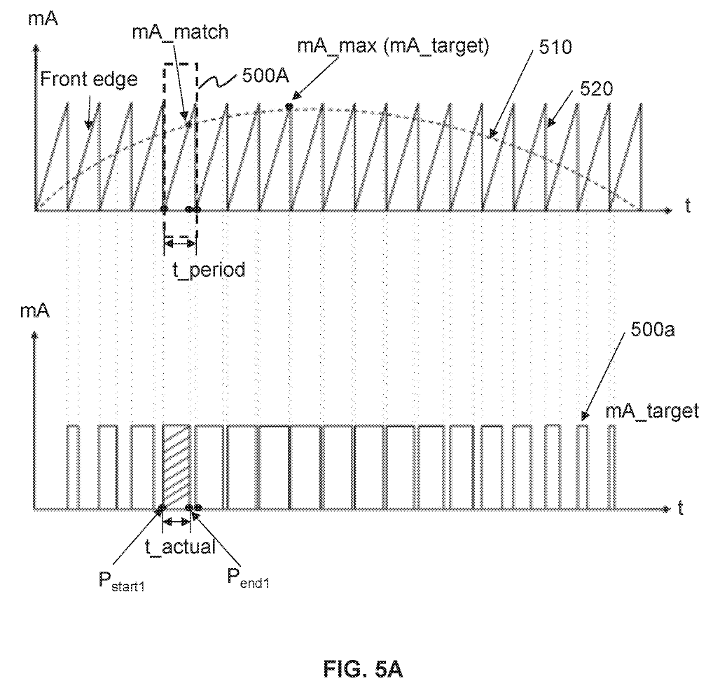

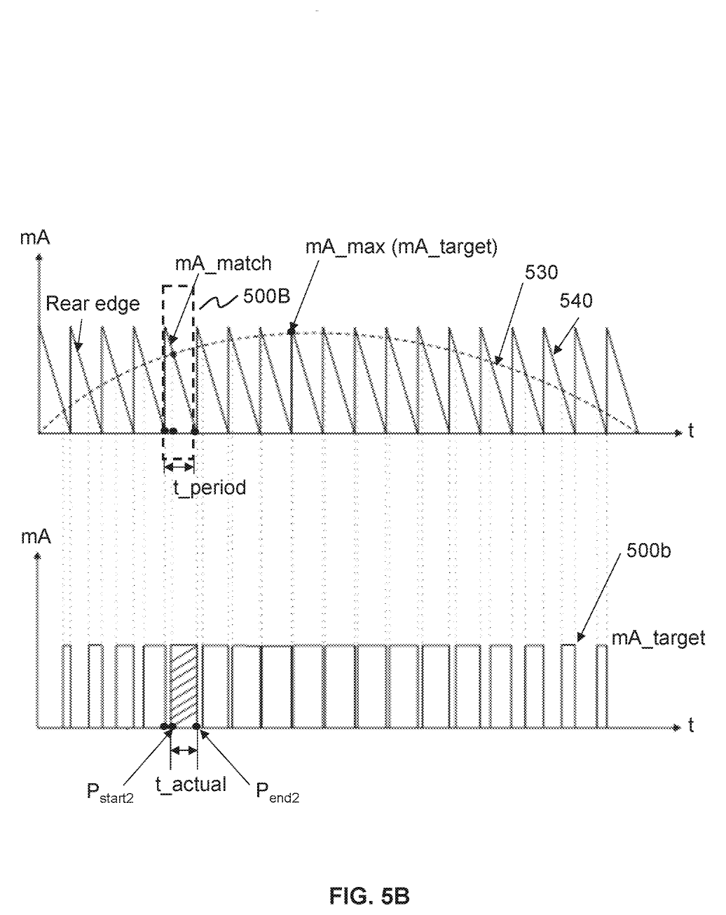

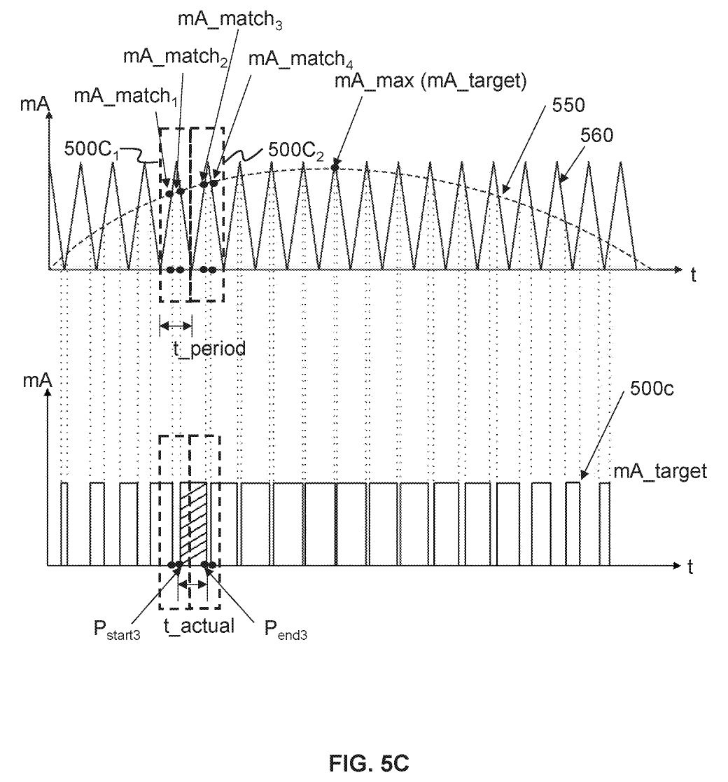

In some embodiments, the triangular shape may be a right triangle (as shown in FIG. 5A or FIG. 5B) or an isosceles triangle (as shown in FIG. 5C). In some embodiments, when the triangular shape is the right triangle, an inclination edge of the right triangle may be a front edge of the right triangle (as shown in FIG. 5A) or a rear edge of the right triangle (as shown in FIG. 5B).

In some embodiments, when the triangular shape is the right triangle and a reset time of the detector can be omitted, i.e., the reset time of the detector is equal to or substantially equal to 0 (e.g., less than a threshold, such as 0.5 μs, 1 μs, 2 μs, 3 μs, 5 μs, etc.), an absolute value of a slope of an inclination edge of the each serration may be determined by dividing the target tube current by the acquisition cycle. In other words, the absolute value of the slope of the inclination edge of the each serration may be equal to a ratio of the target tube current to the corresponding acquisition cycle.

In some embodiments, when the triangular shape is the right triangle and the reset time of the detector can not be omitted, i.e., the reset time of the detector is not equal to 0 (e.g., greater than or equal to the threshold, such as 0.5 μs, 1 μs, 2 μs, 3 μs, 5 μs, etc.) (e.g., the reset time T shown in FIG. 4B), the processing device 120 may determine the absolute value of the slope of the inclination edge of the each serration by dividing the target tube current by a difference between the acquisition cycle and the reset time.

In some embodiments, when the triangular shape is the isosceles triangle and the reset time of the detector can be omitted, i.e., the reset time of the detector is equal to or substantially equal to 0, both a slope of a front edge and an absolute value of a slope of a rear edge of the each serration may be determined by dividing the target tube current by half of the corresponding acquisition cycle. In other words, the slope of the front edge and the absolute value of the slope of the rear edge of the each serration may be both equal to a ratio of the target tube current to half of the corresponding acquisition cycle.

In some embodiments, when the triangular shape is the isosceles triangle and the reset time of the detector can not be omitted, i.e., the reset time of the detector is not equal to 0, the processing device 120 may determine the slope of the front edge and the absolute value of the slope of the rear edge of the each serration by dividing the target tube current by half of a difference between the acquisition cycle and the reset time.

In 430, the processing device 120 (e.g., the power supply control rule determination module 330) may determine, based on the tube current modulation carrier wave and the initial scanning dose curve, a power supply control rule of a grid in an X-ray tube of the X-ray scanning.

The power supply control rule of the grid (also referred to as a grid power supply control rule) may be used to control the power supply for the grid to control the output of the tube current of the X-ray tube.

The power supply control rule may be represented in a form of a pulse curve throughout the entire scanning process. In this form, the amplitude of the pulse curve may remain constant, but the pulse width and pulse interval of the pulse curve may be different. Specifically, the amplitude of the pulse curve may correspond to the output tube current (i.e., the target tube current) of the X-ray tube, and the pulse width may correspond to an emission time period of the X-ray tube. Different pulse widths and pulse intervals result in different emission time periods. Therefore, based on the pulse curve, the radiation dose of the X-ray tube can be controlled to achieve dynamic adjustment of the radiation dose.

In some embodiments, the processing device 120 may determine the power supply control rule based on amplitudes of the tube current modulation carrier wave and the initial scanning dose curve. Specifically, the processing device 120 may determine a period in each acquisition cycle in which an amplitude of the initial scanning dose curve is greater than or equal to an amplitude of the tube current modulation carrier wave as a first target period. It should be noted that an amplitude of a wave refers to a value of a certain point on the wave in the vertical axis direction. The processing device 120 may determine a period in the acquisition cycle in which an amplitude of the initial scanning dose curve is less than an amplitude of the tube current modulation carrier wave as a second target period. In some embodiments, when the reset time of the detector is equal to or substantially equal to 0, the first target period may also be referred to as an emission time period, and the second target period may also be referred to as a non-emission time period. In such cases, the non-emission time period may only include the second target period. In some embodiments, when the reset time of the detector is not equal to 0, the processing device 120 may determine a difference (also referred to as a third target period) between the first target period and the reset time of the detector as an emission time period. The processing device 120 may determine a sum (also referred to as a fourth target period) of the second target period and the reset time of the detector as the non-emission time period. In such cases, the emission time period is a portion of the first target period, and the non-emission time period may not only include the second target period, but also include the reset time of the detector. As a result, the processing device 120 may determine the power supply control rule based on the emission time period and the non-emission time period in the each acquisition cycle.

In some embodiments, the processing device 120 may determine one or more intersection points between the tube current modulation carrier wave and the initial scanning dose curve. The processing device 120 may determine the power supply control rule based on the one or more intersection points and the corresponding time points of the one or more intersection points.

Specifically, in some embodiments, when the triangular shape in each serration of the tube current modulation carrier wave is the right triangle and the inclination edge of the right triangle is the front edge of the right triangle (as shown in FIG. 5A), for each serration, the processing device 120 may first determine an interaction point between the initial scanning dose curve and the front edge of the serration in the tube current modulation carrier wave. The processing device 120 may determine a starting time point of the corresponding acquisition cycle as a starting time point of the emission time period in the corresponding acquisition cycle. The processing device 120 may determine a time point corresponding to the interaction point as an ending time point of the emission time period in the corresponding acquisition cycle. The processing device 120 may determine a time period other than the emission time period in the corresponding acquisition cycle as the non-emission time period.

In some embodiments, when the triangular shape in each serration of the tube current modulation carrier wave is the right triangle and the inclination edge of the right triangle is the rear edge of the right triangle (as shown in FIG. 5B), for each serration, the processing device 120 may also first determine an interaction point between the initial scanning dose curve and the rear edge of the serration in the tube current modulation carrier wave. The processing device 120 may determine a time point corresponding to the interaction point as a starting time point of an emission time period in the corresponding acquisition cycle. The processing device 120 may determine an ending time point of the serration in the corresponding acquisition cycle as an ending time point of the emission time period in the corresponding acquisition cycle. The processing device 120 may determine a time period other than the emission time period in the corresponding acquisition cycle as the non-emission time period.

In some embodiments, when the triangular shape in each serration of the tube current modulation carrier wave is the isosceles triangle (as shown in FIG. 5C). For a first serration, the processing device 120 may determine two interaction points (including a first interaction point and a second interaction point sorted by time) between the initial scanning dose curve and the serration in the tube current modulation carrier wave. Similarly, for a second serration adjacent to the first serration, the processing device 120 may also determine two interaction points (including a third interaction point and a fourth interaction point sorted by time) between the initial scanning dose curve and the second serration in the tube current modulation carrier wave. The processing device 120 may determine a time point corresponding to the second interaction point as a starting time point of the emission time period. The processing device 120 may determine a time point corresponding to the third interaction point as an ending time point of the emission time period. The processing device 120 may determine a time period between a time point corresponding to the first interaction point and the time point corresponding to the second interaction point as the non-emission time period. The processing device 120 may also determine a time period between a time point corresponding to the third interaction point and the time point corresponding to the forth interaction point as another non-emission time period.

More descriptions for the determining the grid power supply control rule may be found elsewhere in the present disclosure, e.g., in FIG. 5A to FIG. 5C and the descriptions thereof.

In 440, the processing device 120 (e.g., the control module 340) may control a high-voltage generator to supply power to the grid according to the power supply control rule to control an output state of tube current of the X-ray tube.

The output state of tube current may characterize whether the X-ray tube has a tube current output. When the X-ray tube has a tube current output, an output value of the tube current may be equal to or substantially equal to the target tube current.

The grid may be disposed at a cathode filament of the X-ray tube. Specifically, by controlling the high-voltage generator to turn on the grid according to the power supply control rule, electrons emitted from the cathode filament of the X-ray tube may be deflected and unable to impinge on a small area (i.e., a focus) on the anode target of the X-ray tube to generate radiation beams (X-rays), resulting in no tube current output from the X-ray tube. On the contrary, by controlling the high-voltage generator to turn off the grid according to the power supply control rule, electrons emitted from the cathode filament of the X-ray tube may be impinged on the small area on the anode target of the X-ray tube to generate radiation beams (X-rays), resulting in tube current output from the X-ray tube.

In some embodiments, during each emission time period in the power supply control rule, the processing device 120 may control the high-voltage generator to provide a first level to the grid to turn off the grid. In such cases, the X-ray tube may continuously output the target tube current during the emission time period. Further, during the non-emission time period in the power supply control rule, the processing device 120 may provide a second level to the grid to turn on the grid. In such cases, the X-ray tube may not output the tube current, but the filament temperature of the X-ray tube remains constant. Since a control speed of the grid is generally in microseconds or tens of microseconds, which has an order of magnitude improvement in control effect compared to filament temperature control, the radiation dose can quickly and accurately follow changes in a target dose.

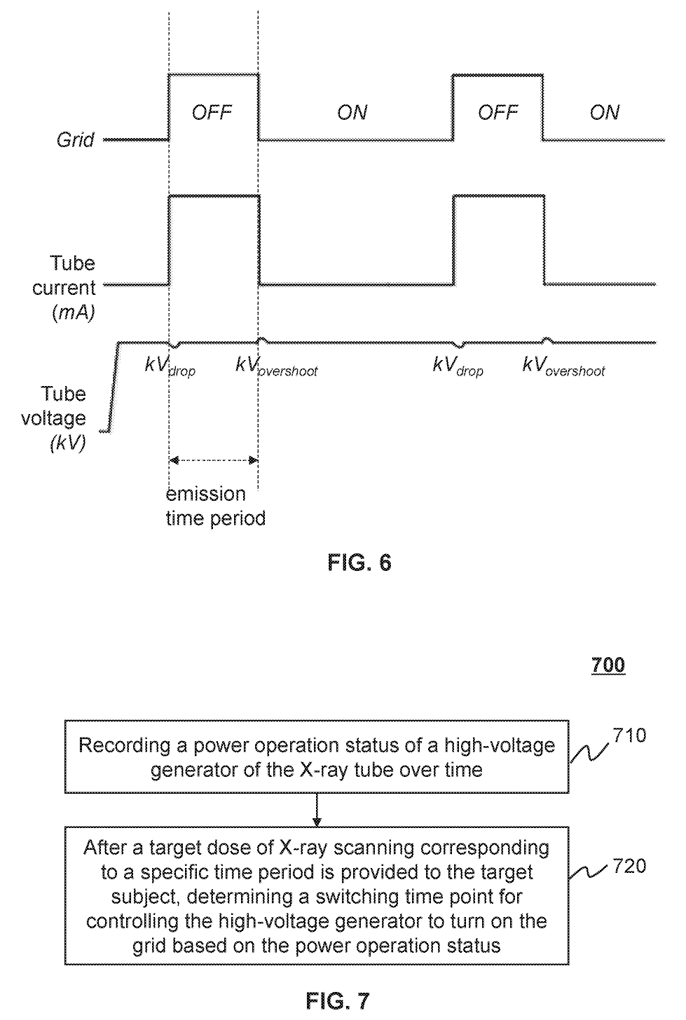

In some embodiments, as shown in FIG. 6, during the process (i.e., a dose modulation process) for controlling a high-voltage generator to supply power to the grid according to the power supply control rule to control the output state of tube current of the X-ray tube, when controlling the high-voltage generator to turn on or turn off the grid, there may be a tube voltage drop (e.g., kV_{drop}) or overshoot (e.g., kVₒᵥₑᵣₛₕₒₒₜ) in the tube voltage, which may cause voltage fluctuations and affect the accuracy of dose modulation.

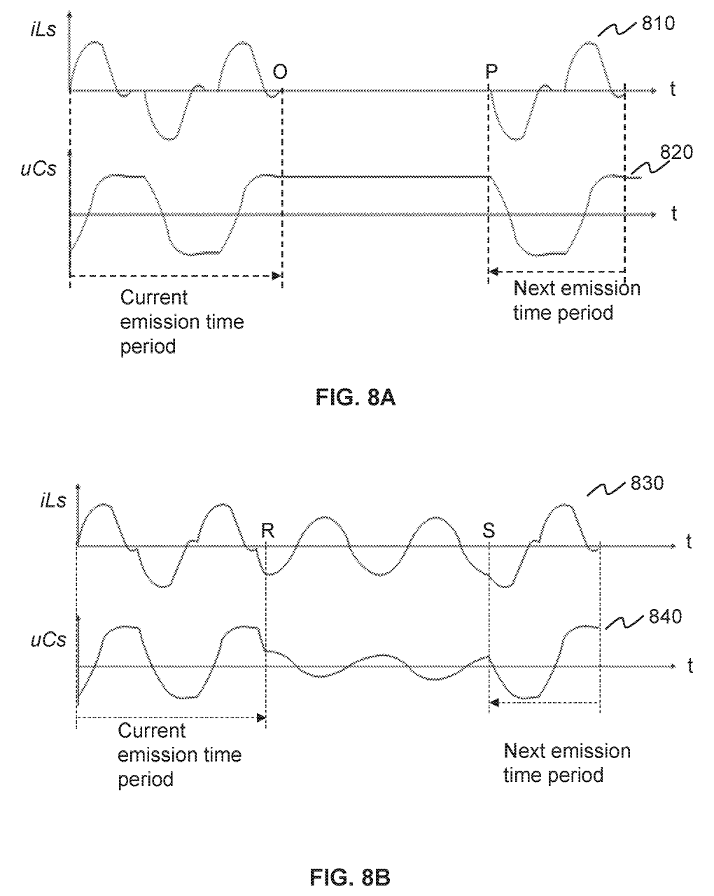

Thus, in order to ensure stable tube voltage during the dose modulation process to the accuracy of dose modulation, the processing device 120 may record a power operation status of the high-voltage generator of the X-ray tube over time. As used herein, the power operation status of the high-voltage generator may be characterized by electrical parameters of the high-voltage generator, such as a resonant inductor current (e.g., the resonant inductor current curve 810 shown in FIG. 8A) and/or resonant capacitor voltage (e.g., the resonant capacitor voltage curve 820 shown in FIG. 8A) of the high-voltage generator. After a target dose of X-ray scanning corresponding to a current emission time period is provided to the target subject, the processing device 120 may determine a switching time point for controlling the high-voltage generator to turn on the grid based on the power operation status. For example, when the high-voltage generator operates in a discontinuous current mode, the switching time point may correspond to a time point when a resonant inductor current in the high-voltage generator is 0, e.g., the time point O in FIG. 8A. As a result, compared to a switching time point (i.e., the ending point of the current emission time period) directly determined from the power supply control rule to turn on the grid, the turning on of the grid is delayed. As another example, when the high-voltage generator operates in a continuous current mode, the switching time point may correspond to a time point after the target dose of the emission time period is provided to the target subject. That is, the switching time point may be the ending time point of the emission time period.

Further, the processing device 120 may simultaneously record a power modulation status (e.g., frequency, duty ratio, etc.) of the high-voltage generator at the switching time point. The processing device 120 may determine the power modulation status of the high-voltage generator loaded at the starting time point (e.g., the time point P in FIG. 8A) of the next emission time period based on the recorded power modulation status of the high-voltage generator at the switching time point. It should be noted that when using the high-voltage generator to supply power, the output of the high-voltage generator may be in a power modulation mode including a pulse width modulation (PWM) mode, a pulse frequency modulation (PFM) mode, a pulse density modulation (PDM) mode, or the like, or any combination thereof. The power modulation status is a status of the power modulation mode of the high-voltage generator at a certain time point.

More descriptions for the controlling of the high-voltage generator to supply power to the grid according to the power supply control rule may be found elsewhere in the present disclosure, e.g., in FIG. 7 and the descriptions thereof.

According to some embodiments of the present disclosure, by generating the power supply control rule based on the tube current modulation carrier wave and the initial scanning dose curve, and controlling the high-voltage generator to supply power to the grid according to the power supply control rule, the dose modulation of the X-ray scanning can be controlled through the grid using the power supply control rule of the grid, that is, to control the emission time at different scanning positions while keeping an output amplitude of the tube current unchanged to achieve dose modulation, thereby avoiding real-time changes in a filament temperature of the X-ray tube during the scanning process. Further, since a control speed of the grid is generally in microseconds or tens of microseconds, which has an order of magnitude improvement in control effect compared to filament temperature control, the radiation dose can quickly and accurately follow changes in a target dose.

Moreover, the output current of the X-ray tube is equal to the target tube current which is greater than or equal to a maximum tube current in the initial scanning dose curve, which can meet the requirements of image brightness and signal-to-noise ratio of the target subject at different scanning positions (or scanning angles).

FIG. 4C is a flowchart illustrating an exemplary process for dose modulation of an X-ray scanning according to some embodiments of the present disclosure. In some embodiments, process 400C may be executed by the imaging system 100. For example, process 400C may be implemented as a set of instructions (e.g., an application) stored in the storage device 130, and the processing device 120 (e.g., one or more modules in FIG. 3) may execute the set of instructions and may accordingly be directed to perform the process 400C.

In 401, the processing device 120 (e.g., the obtaining module 310) may obtain an initial scanning dose rule for scanning a target subject.

The initial scanning dose rule represents the variation of a tube current of an X-ray tube required for scanning the target subject over time.

In some embodiments, the initial scanning dose rule may include an initial scanning dose curve or a plurality of discrete points. In some embodiments, the initial scanning dose curve may be obtained by fitting the plurality of discrete points. In some embodiments, the initial scanning dose rule may also be represented as a table, a function, etc. The initial scanning dose curve may also be determined based on the table, the function, etc. For illustration purpose, an initial scanning dose curve is taken as an example of the initial scanning dose rule.

In some embodiments, the processing device 120 may obtain the initial scanning dose curve in connection with operation 410 in FIG. 4A, which may not be repeated here.

In 402, the processing device 120 (e.g., the power supply control rule determination module 330) may determine, based on the initial scanning dose rule, a power supply control rule of a grid in an X-ray tube of the X-ray scanning.

The power supply control rule is configured to maintain an output value of the tube current, and change an emission dose of each acquisition cycle of a detector by controlling an emission time period through turning on/off the grid.

In some embodiments, the processing device 120 may further obtain an acquisition cycle of a detector. The processing device 120 may generate a tube current modulation carrier wave at least based on the acquisition cycle and a target tube current. The target tube current may be greater than or equal to a maximum tube current in the initial scanning dose curve (i.e., the initial scanning dose rule). The processing device 120 may determine, based on the tube current modulation carrier wave and the initial scanning dose curve, the power supply control rule of the grid in the X-ray tube of the X-ray scanning. More description for the determining the power supply control rule based on the tube current modulation carrier wave and the initial scanning dose curve can be found elsewhere in the present disclosure (e.g., FIG. 4A and the descriptions thereof).

In some embodiments, the processing device 120 may convert the initial scanning dose curve into a voltage curve representing the variation of voltage over time. The processing device 120 may determine the power supply control rule based on the voltage curve using a pulse width modulation (PWM) technique. Exemplary pulse width modulation techniques may include a sinusoidal PWM (SPWM) technique, a random pulse width (SPWM) modulation technique, etc. A total area formed by the power supply control rule is equal to a total area formed by the initial scanning dose curve.

In 403, the processing device 120 (e.g., the control module 340) may control a high-voltage generator to supply power to the grid according to the power supply control rule to control an output state of tube current of the X-ray tube.

An output value of the tube current is equal to the target tube current as described above. More description for the controlling the high-voltage generator to supply power to the grid according to the power supply control rule can be found elsewhere in the present disclosure (e.g., FIG. 4A and the descriptions thereof).

FIG. 5A is a schematic diagram illustrating an exemplary process for generating a grid power supply control rule when the target tube current is equal to the maximum tube current in the initial scanning dose curve according to some embodiments of the present disclosure.

As shown in FIG. 5A, a curve 510 represents an initial scanning dose curve for scanning a target subject. A curve 520 represents a tube current modulation carrier wave, which is a sawtooth wave including a plurality of serrations. The triangular shape in each serration of the tube current modulation carrier wave 520 is a right triangle and an inclination edge of the right triangle is the front edge of the right triangle. A maximum tube current (i.e., a target tube current mA_target) in the tube current modulation carrier wave 520 is equal to a maximum tube current mA_max in the initial scanning dose curve 510. As a result, the target tube current mA_target is equal to the maximum tube current mA_max in the initial scanning dose curve 510.

For each serration, e.g., the serration 500A of the tube current modulation carrier wave 520, during a process for determining a grid power supply control rule (i.e., the pulse curve 500a shown in FIG. 5A), the processing device 120 may first determine an interaction point mA_match between the initial scanning dose curve 510 and the inclination edge of the serration 500A in the tube current modulation carrier wave 520. The processing device 120 may determine a starting time point Pₛₜₐᵣₜ₁ of the corresponding acquisition cycle t_period as a starting time point Pₛₜₐᵣₜ₁ of an emission time period t_actual in the corresponding acquisition cycle. The processing device 120 may determine a time point corresponding to the interaction point as an ending time point P_{end1} of the emission time period t_actual in the corresponding acquisition cycle t_period. The processing device 120 may determine a time period other than the emission time period t_actual in the corresponding acquisition cycle t_period as a non-emission time period.

Therefore, the determined grid power supply control rule may be a pulse curve 500a with the same amplitude, different pulse widths, and different pulse intervals. Specifically, the amplitude of the pulse curve 500a may correspond to an output tube current (i.e., the target tube current) of the X-ray tube, and the pulse width may correspond to the emission time of the X-ray tube.

FIG. 5B is a schematic diagram illustrating an exemplary process for generating a grid power supply control rule when the target tube current is equal to the maximum tube current in the initial scanning dose curve according to some embodiments of the present disclosure.

As shown in FIG. 5B, a curve 530 represents an initial scanning dose curve for scanning a target subject. A curve 540 represents a tube current modulation carrier wave, which is a sawtooth wave including a plurality of serrations. The triangular shape in each serration of the tube current modulation carrier wave 540 is a right triangle and an inclination edge of the right triangle is the rear edge of the right triangle. A maximum tube current mA_max in the initial scanning dose curve 530 is equal to a maximum tube current in the tube current modulation carrier wave 540. As a result, the target tube current mA_target is equal to the maximum tube current mA_max in the initial scanning dose curve 530.

For each serration, e.g., the serration 500B of the tube current modulation carrier wave 540, during a process for determining a grid power supply control rule (i.e., the pulse curve 500b shown in FIG. 5B), the processing device 120 may first determine an interaction point mA_match between the initial scanning dose curve 530 and the inclination edge of the serration 500B in the tube current modulation carrier wave 540. The processing device 120 may determine a time point corresponding to the interaction point mA_match as a starting time point Pₛₜₐᵣₜ₂ of an emission time period t_actual in the corresponding acquisition cycle. The processing device 120 may determine an ending time point of the corresponding acquisition cycle t_period as an ending time point P_{end2} of the emission time period t_actual in the corresponding acquisition cycle t_period. The processing device 120 may determine a time period other than the emission time period t_actual in the corresponding acquisition cycle t_period as a non-emission time period.

Therefore, the determined grid power supply control rule may be a pulse curve 500b with different pulse widths and pulse intervals. Specifically, the amplitude of the pulse curve 500b may correspond to an output tube current (i.e., the target tube current) of the X-ray tube, and the pulse width may correspond to the emission time of the X-ray tube.

FIG. 5C is a schematic diagram illustrating an exemplary process for generating a grid power supply control rule when the target tube current is equal to the maximum tube current in the initial scanning dose curve according to some embodiments of the present disclosure.

As shown in FIG. 5C, curve 550 represents an initial scanning dose curve for scanning a target subject. Curve 560 represents a tube current modulation carrier wave, which is a sawtooth wave including a plurality of serrations. The triangular shape in each serration of the tube current modulation carrier wave 560 is an isosceles triangle. A maximum tube current mA_max in the initial scanning dose curve 550 is equal to a maximum tube current in the tube current modulation carrier wave 560. As a result, the target tube current mA_target is equal to the maximum tube current mA_max in the initial scanning dose curve 550.

For each serration, e.g., the serration 500C₁ of the tube current modulation carrier wave 560, during a process for determing a grid power supply control rule (i.e., the pulse curve 500c shown in FIG. 5C), the processing device 120 may determine two interaction points mA_match₁ and mA_match₂ between the initial scanning dose curve 550 and the serration 500C₁ in the tube current modulation carrier wave 560. Similarly, for the serration 500C₂, the processing device 120 may also determine two interaction points mA_match₃ and mA_match₄ between the initial scanning dose curve 550 and the serration 500C₂ in the tube current modulation carrier wave 560. The processing device 120 may determine a time point corresponding to the interaction point mA_match₂ as a starting time point Pₛₜₐᵣₜ₂ of an emission time period t_actual. Further, the processing device 120 may determine a time point corresponding to the interaction point mA_match₃ as an ending time point P_{end3} of the emission time period t_actual. The processing device 120 may determine a time period between a time point corresponding to the interaction point mA_match₁ and the time point corresponding to the interaction point mA_match₂ (i.e., the starting time point Pₛₜₐᵣₜ₃) as a non-emission time period. The processing device 120 may also determine a time period between a time point corresponding to the interaction point mA_match₃ (i.e., the starting time point P_{end3}) and the time point corresponding to the interaction point mA_match₄ as another non-emission time period.

Therefore, the determined grid power supply control rule may be a pulse curve 500c with different pulse widths and pulse intervals. Specifically, the amplitude of the pulse curve 500c may correspond to an output tube current (i.e., the target tube current) of the X-ray tube, and the pulse width may correspond to the emission time of the X-ray tube.

FIG. 6 is a diagram illustrating exemplary variation curves of tube voltage and tube current when turning on/off a grid according to some embodiments of the present disclosure.

As shown in FIG. 6, at the beginning of a certain emission time period, the grid is turned off, and the tube current starts outputting. At this time, the high-voltage generator enters a working load state from a no-load state. The tube voltage may experience a certain drop, with a drop amount of kV_{drop}. After a target dose corresponding to the certain emission time period, i.e., the ending time point of the certain emission time period is reached, the grid may be turned on, and the tube current may stop from outputting. At this time, the high-voltage generator enters the no-load state from the working load state. The tube voltage may experience a certain overshoot, with an overshoot amount of kVₒᵥₑᵣₛₕₒₒₜ. The changes (i.e., the drop with the drop amount of kV_{drop}, and the overshoot with the overshoot amount of kVₒᵥₑᵣₛₕₒₒₜ) in tube voltage during the above two stages may affect an X-ray energy spectrum of X-rays, which may have an impact on the final imaging effect.

In order to ensure stable tube voltage during the dose modulation process to the accuracy of dose modulation, a power adjustment strategy is provided in FIG. 7.

FIG. 7 is a flowchart illustrating an exemplary process for controlling a high-voltage generator to supply power to a grid according to a power supply control rule according to some embodiments of the present disclosure. In some embodiments, the process for controlling the high-voltage generator to supply power to the grid as described in connection with operation 440 in FIG. 4A may be performed according to the process 700.

In 710, the processing device 120 (e.g., the control module 340) may record a power operation status of the high-voltage generator for the X-ray tube over time.

As used herein, the power operation status of the high-voltage generator may be characterized by electrical parameters of the high-voltage generator, such as a resonant inductor current and/or resonant capacitor voltage of the high-voltage generator. For example, the processing device 120 may record a resonant inductor current curve (e.g., the curve 810 in FIG. 8A) and a resonant capacitor voltage curve (e.g., the curve 820 in FIG. 8A) of the high-voltage generator.

In 720, after a target dose of X-ray scanning corresponding to a current emission time period is provided to the target subject (i.e., corresponding to a time point when the high-voltage generator enters the no-load state from the working load state), the processing device 120 (e.g., the control module 340) may determine a target switching time point for controlling the high-voltage generator to turn on the grid based on the power operation status. In some embodiments, the current emission time period may be an emission time period in a power supply control rule as described in FIG. 4A. The target dose may be determined based on the emission time period and the amplitude in the power supply control rule.

In some embodiments, when the high-voltage generator operates in a discontinuous current mode, that is, a value of current in the high-voltage generator may be equal to 0 within a certain time period, the processing device 120 may record the variation of the resonant inductor current over time starting from the ending time point of the current emission time period. The processing device 120 may determine a time point at which the resonant inductor current in the high-voltage generator becomes 0 state as the target switching time point for controlling the high-voltage generator to turn on the grid (also be referred to as switching the high-voltage generator from the working load state to the no-load state). That is to say, relative to the ending time point of the current emission time period, the target switching time point may be delayed, i.e., the turning on of the grid may be delayed.

In some embodiments, in order to further avoid voltage fluctuations in the tube voltage, the processing device 120 may simultaneously record a power modulation status of the high-voltage generator at the target switching time point. The processing device 120 may control the high-voltage generator to turn on the grid at the target switching time point. Further, the processing device 120 may control the high-voltage generator to turn off the grid (also be referred to as switching the high-voltage generator from the no-load state to the working load state) at a starting point (also referred to as a first switching time point) of a next emission time period next to the current emission time period by providing the power modulation status of the high-voltage generator at the target switching time point to the high-voltage generator.

FIG. 8A is a diagram illustrating a resonant inductor current curve and a resonant capacitor voltage curve of a high-voltage generator when the high-voltage generator operates in a discontinuous current mode according to some embodiments of the present disclosure. As illustrated in FIG. 8A, a curve 810 represents a resonant inductor current curve (iLs) of the high-voltage generator, and a curve 820 represents a resonant capacitor voltage curve (uCs) of the high-voltage generator. Merely by way of example, for a current emission time period, a target dose corresponding to the current emission time period may be completely provided to the target subject before point O. After the target dose corresponding to the current emission time period is provided to the target subject, the processing device 120 may not immediately control the high-voltage generator to turn on the grid, but may control the high-voltage generator to turn on the grid when the resonant inductor current in the high-voltage generator first becomes 0 (i.e., point O). The processing device 120 may record the resonant capacitor voltage at the point O. When a next emission time period arrives, the processing device 120 may control the high-voltage generator to turn off the grid immediately, i.e., the grid is turned off at the starting time point (i.e., point P) of the next emission time period. At this time point, the processing device 120 may load the power modulation status of the high-voltage generator at point O to point P. In other words, the power modulation status of the high-voltage generator at point O is the same as the power modulation status of the high-voltage generator at point P.

In some embodiments, when the high-voltage generator operates in a continuous current mode, the processing device 120 may begin to record the resonant inductor current at the ending time point of the current emission time period. The processing device 120 may determine the ending time point of the current emission time period as the target switching time point for controlling the high-voltage generator to turn on the grid, that is, the grid may be turned on immediately.

In some embodiments, in order to further avoid voltage fluctuations in the tube voltage, the processing device 120 may simultaneously record the power modulation status and a target resonant inductor current of the high-voltage generator at the target switching time point (i.e., the ending time point of the current emission time period). The processing device 120 may control the high-voltage generator to turn on the grid at the target switching time point. Further, when a next emission time period arrives, the processing device 120 may control the high-voltage generator to turn off the grid at a time point (also referred to as a second switching time point) when the resonant inductor current first becomes to be the same as the target resonant inductor current at the target switching time point. That is to say, relative to the starting time point of the next emission time period, the actual switching time point may be delayed, i.e., the turning off of the grid in the next emission time period may be delayed.

FIG. 8B is a diagram illustrating a resonant inductor current curve and a resonant capacitor voltage curve of a high-voltage generator when the high-voltage generator operates in a continuous current mode according to some embodiments of the present disclosure. As illustrated in FIG. 8B, a curve 830 represents a resonant inductor current curve (iLs) of the high-voltage generator, and a curve 840 represents a resonant capacitor voltage curve (uCs) of the high-voltage generator. Merely by way of example, for a current emission time period, a target dose corresponding to the current emission time period may be completely provided to the target subject at point R. After the target dose corresponding to the current emission time period is provided to the target subject, the processing device 120 may immediately control the high-voltage generator to turn on the grid, i.e., at point R. The processing device 120 may record the resonant capacitor voltage and the resonant inductor current at the point R. When a next emission time period arrives, the processing device 120 may not control the high-voltage generator to turn off the grid immediately, but may control the high-voltage generator to turn off the grid at the time point (i.e., point S) when the resonant inductor current at point S is the same as the resonant inductor current at point R. At this time point, the processing device 120 may load the power modulation status of the high-voltage generator at point R to point S. In other words, the power modulation status of the high-voltage generator at point R is the same as the power modulation status of the high-voltage generator at point S.

It should be noted that the power supply control rule for controlling the high-voltage generator to switch the high-voltage generator from the working load state to the no-load state (or from the no-load state to the working load state) can be any power supply control rule (e.g., the power supply control rule of the grid in the X-ray tube) that can provide an initial switching time point (e.g., a first initial switching time point for switching the high-voltage generator from the working load state to the no-load state, or a second initial switching time point for switching the high-voltage generator from the no-load state to the working load state).

According to some embodiments of the present disclosure, after a target dose of X-ray scanning corresponding to a current emission time period is provided to the target subject, by determining the target switching time point for controlling the high-voltage generator to turn on the grid based on the power operation status of the high-voltage generator, and turn off the grid at another switching time point in the next emission time period by providing the high-voltage generator with a power modulation status that is the same as the power modulation status of the high-voltage generator at the target switching time point, the high-voltage generator can quickly and stably switch to workload operation, avoiding tube voltage fluctuations.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Further, a non-transitory computer-readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer-readable signal medium may be any computer-readable medium that is not a computer-readable storage medium and that may communicate, propagate, or transport a program for use by or in connection with an instruction execution system, apparatus, or device. Program code embodied on a computer-readable signal medium may be transmitted using any appropriate medium, including wireless, wireline, optical fiber cable, RF, or the like, or any suitable combination of the foregoing.

In some embodiments, the numbers expressing quantities, properties, and so forth, used to describe and claim certain embodiments of the application are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

What is claimed is:

1. A system for dose modulation of an X-ray scanning, comprising:
at least one storage device storing executable instructions; and
at least one processor in communication with the at least one storage device, wherein when executing the executable instructions, the at least one processor is configured to cause the system to perform operations including:
obtaining, for scanning a target subject, an acquisition cycle of a detector and an initial scanning dose curve;
generating a tube current modulation carrier wave at least based on the acquisition cycle and a target tube current, the target tube current being greater than or equal to a maximum tube current in the initial scanning dose curve;
determining, based on the tube current modulation carrier wave and the initial scanning dose curve, a power supply control rule of a grid in an X-ray tube of the X-ray scanning; and
controlling a high-voltage generator to supply power to the grid according to the power supply control rule to control an output state of tube current of the X-ray tube, wherein an output value of the tube current is equal to the target tube current.

2. The system of claim 1, wherein the tube current modulation carrier wave is a sawtooth wave including a plurality of serrations, each serration being in a triangular shape in each acquisition cycle.

3. The system of claim 2, wherein the triangular shape is a right triangle or an isosceles triangle.

4. The system of claim 3, wherein when the triangular shape is the right triangle and a reset time of the detector is equal to 0, an absolute value of a slope of an inclination edge of the each serration is equal to a ratio of the target tube current to the corresponding acquisition cycle.

5. The system of claim 3, wherein when the triangular shape is the isosceles triangle and a reset time of the detector is equal to 0, a slope of a front edge and an absolute value of a slope of a rear edge of the each serration are both equal to a ratio of the target tube current to half of the corresponding acquisition cycle.

6. The system of claim 4, wherein the controlling a high-voltage generator to supply power to the grid of the X-ray tube according to the power supply control rule includes:
during a first target period of each acquisition cycle, controlling the high-voltage generator to provide a first level to the grid to turn off the grid; and
during a second target period of the acquisition cycle, controlling the high-voltage generator to provide a second level to the grid to turn on the grid, wherein
the first target period corresponds to a period in the acquisition cycle in which an amplitude of the initial scanning dose curve is greater than or equal to an amplitude of the tube current modulation carrier wave, and
the second target period corresponds to a period in the acquisition cycle in which an amplitude of the initial scanning dose curve is less than an amplitude of the tube current modulation carrier wave.

7. The system of claim 3, wherein when the triangular shape is the right triangle and a reset time of the detector is not equal to 0, an absolute value of a slope of an edge of the each serration is equal to a ratio of the target tube current to a difference between the corresponding acquisition cycle and the reset time.

8. The system of claim 3, wherein when the triangular shape is the isosceles triangle and a reset time of the detector is not equal to 0, a slope of a front edge and an absolute value of a slope of a rear edge of the each serration are both equal to a ratio of the target tube current to half of a difference between the acquisition cycle and the reset time.

9. The system of claim 7, wherein the controlling a high-voltage generator to supply power to the grid of the X-ray tube according to the power supply control rule includes:
during a third target period of each acquisition cycle, controlling the high-voltage generator to provide a first level to the grid to turn off the grid; and
during a fourth target period of the acquisition cycle, controlling the high-voltage generator to provide a second level to the grid to turn on the grid, wherein
the third target period is a difference between a first time period and the reset time of the detector, wherein the first target period corresponds to a period in the acquisition cycle in which an amplitude of the initial scanning dose curve is greater than or equal to an amplitude of the tube current modulation carrier wave, and
the fourth target period is a sum of a second target period and the reset time of the detector, wherein the second target period corresponds to a period in the acquisition cycle in which an amplitude of the initial scanning dose curve is less than an amplitude of the tube current modulation carrier wave.

10. The system of claim 1, wherein the controlling a high-voltage generator to supply power to the grid of the X-ray tube according to the power supply control rule includes:
recording a power operation status of the high-voltage generator of the X-ray tube over time; and
after a target dose of X-ray scanning corresponding to a current emission time period is provided to the target subject, determining a target switching time point for controlling the high-voltage generator to turn on the grid based on the power operation status, the current emission time period is determined based on the power supply control rule.

11. The system of claim 10, wherein when the high-voltage generator operates in a discontinuous current mode, the target switching time point corresponds to a time point when a resonant inductor current in the high-voltage generator is 0.

12. The system of claim 11, wherein the at least one processor is further configured to cause the system to perform operations including:
simultaneously recording a power modulation status of the high-voltage generator at the target switching time point; and
controlling the high-voltage generator to turn off the grid at a first switching time point by providing the power modulation status of the high-voltage generator at the target switching time point to the high-voltage generator, wherein
the first switching time point is a time point corresponding to a starting point of an emission time period next to the current emission time period for controlling the high-voltage generator to turn off the grid.

13. The system of claim 10, wherein when the high-voltage generator operates in a continuous current mode, the target switching time point corresponds to a time point after the target dose of the specific time period is provided to the target subject.

14. The system of claim 13, wherein the at least one processor is further configured to cause the system to perform operations including:
simultaneously recording a power modulation status and a target resonant inductor current of the high-voltage generator at the target switching time point; and
controlling the high-voltage generator to turn off the grid at a second switching time point by providing the power modulation status of the high-voltage generator at the switching time point to the high-voltage generator, wherein
the second switching time point is a time point when a resonant inductor current of the high-voltage generator is equal to the target resonant inductor current that is after a starting point of an emission time target period next to the current emission time period for controlling the high-voltage generator to turn off the grid.

15. A system for power adjustment, comprising:
at least one storage device storing executable instructions; and
at least one processor in communication with the at least one storage device, wherein when executing the executable instructions, the at least one processor is configured to cause the system to perform operations including:
obtaining a power supply control rule for controlling a high-voltage generator;
determining a first initial switching time point for switching a high-voltage generator from a working load state to a no-load state based on the power supply control rule,
if the high-voltage generator operates in a discontinuous current mode,
controlling the high-voltage generator to switch from the working load state to the no-load state at a first target switching time point when a resonant inductor current in the high-voltage generator is 0; or
if the high-voltage generator operates in a continuous current mode,
controlling the high-voltage generator to switch from the working load state to the no-load state at the first initial switching time point.

16. The system of claim 15, wherein the at least one processor is further configured to cause the system to perform operations including:
determining a second initial switching time point for switching the high-voltage generator from the no-load state to the working load state based on the power supply control rule,
if the high-voltage generator operates in the discontinuous current mode,
obtaining a power modulation status of the high-voltage generator at the first target switching time point, and
controlling the high-voltage generator to switch from the no-load state to the working load state at the second initial switching time point by providing the power modulation status of the high-voltage generator at the first target switching time point to the high-voltage generator; or
if the high-voltage generator operates in the continuous current mode,
obtaining a power modulation status and a target resonant inductor current of the high-voltage generator at the first initial switching time point, and
controlling the high-voltage generator to switch from the no-load state to the working load state at a second target switching time point by providing the power modulation status of the high-voltage generator at the first initial switching time point to the high-voltage generator, wherein the second target switching time point corresponds to a time point when the resonant inductor current of the high-voltage generator is equal to the target resonant inductor current of the high-voltage generator at the first initial switching time point.

17. A system for dose modulation of an X-ray scanning, comprising:
at least one storage device storing executable instructions; and
at least one processor in communication with the at least one storage device, wherein when executing the executable instructions, the at least one processor is configured to cause the system to perform operations including:
obtaining an initial scanning dose rule for scanning a target subject;
determining, based on the initial scanning dose rule, a power supply control rule of a grid in an X-ray tube of the X-ray scanning; and
controlling a high-voltage generator to supply power to the grid according to the power supply control rule to control an output state of tube current of the X-ray tube.

18. The system of claim 17, wherein the power supply control rule is configured to maintain an output value of the tube current, and change an emission dose of each acquisition cycle of a detector by controlling an emission time period through turning on/off the grid.

19. The system of claim 17, wherein the initial scanning dose rule includes an initial scanning dose curve or a plurality of discrete points.

20. The system of claim 19, wherein the initial scanning dose rule is the initial scanning dose curve, the determining, based on the initial scanning dose rule, a power supply control rule of a grid in an X-ray tube of the X-ray scanning includes:
generating a tube current modulation carrier wave at least based on an acquisition cycle of the detector and a target tube current, the target tube current being greater than or equal to a maximum tube current in the initial scanning dose curve; and
determining, based on the tube current modulation carrier wave and the initial scanning dose curve, the power supply control rule.
